(19) United States
(12) Patent Application Publication
(10) Pub. No.: US 2026/0152459 A1
(43) Pub. Date: Jun. 4, 2026

(54) METHOD FOR PRODUCING (POLY)ALKYLENE GLYCOL MONOALKYL ETHER

(71) Applicant: NIPPON SHOKUBAI CO., LTD., Osaka (JP)

(72) Inventors: Akiko YAMAUCHI, Kawasaki-shi, Kanagawa (JP); Hiroki KATAOKA, Kawasaki-shi, Kanagawa (JP); Toru INAOKA, Kawasaki-shi, Kanagawa (JP); Yudai FUJII, Kawasaki-shi, Kanagawa (JP); Takeo AKATSUKA, Kawasaki-shi, Kanagawa (JP); Masaru KIRISHIKI, Suita-shi, Osaka (JP)

(21) Appl. No.: 19/119,505

(22) PCT Filed: Oct. 20, 2023
(86) PCT No.: PCT/JP2023/038099

§ 371 Date: Apr. 9, 2025

(30) Foreign Application Priority Data
Oct. 21, 2022 (JP) 2022-169504

(51) Int. Cl. C07C 43/13; C07C 41/09; C07C 41/42; C08G 65/26

(52) U.S. Cl. CPC C07C 43/13; C07C 41/09; C07C 41/42; C08G 65/2609; C08G 65/2657

(57) ABSTRACT

A method for producing a (poly)alkylene glycol monoalkyl ether includes reacting an olefin and a (poly)alkylene glycol in a reactor in a presence of a catalyst. The method also includes recovering at least a portion of raw materials used for the production to use the recovered raw materials again as a raw material, where at least one of the recovered raw materials contains an olefin. A mass of a branched olefin relative to a mass sum of the branched olefin and a linear olefin contained in the recovered raw materials is controlled not to exceed 20 mass %.

### TECHNICAL FIELD

The present invention relates to a method for producing a (poly)alkylene glycol monoalkyl ether.

### BACKGROUND ART

In the related art, there has been known a method for efficiently producing a (poly)alkylene glycol monoalkyl ether by reacting an olefin with a (poly)alkylene glycol in the presence of a catalyst.

For example, Patent Document 1 (JP H10-218819) discloses a production method in which a catalyst is partially regenerated and used again as a catalyst to produce a (poly)alkylene glycol monoalkyl ether with high selectivity and high yield. This tackles the following phenomenon: when unreacted raw materials are recycled to continuously produce the (poly)alkylene glycol monoalkyl ether, the catalytic activity is lowered due to an increase in operation time to lower the yield of the product.

Patent Document 2 (JP H10-168016) discloses a method for producing a (poly)alkylene glycol monoalkyl ether with high selectivity and high yield by recovering a (poly)alkylene glycol dialkyl ether and/or alcohol by-produced in a reaction of a (poly)alkylene glycol monoalkyl ether, supplying the recovered (poly)alkylene glycol dialkyl ether and/or alcohol to a reaction system, and reacting an olefin with a (poly)alkylene glycol in the presence of the recovered (poly)alkylene glycol dialkyl ether and/or alcohol.

### SUMMARY OF INVENTION

Although the (poly)alkylene glycol monoalkyl ether can be continuously produced by the production methods in the related art, there is still a problem in that the yield of the (poly)alkylene glycol monoalkyl ether per hour is lowered due to the reaction for a long time.

Accordingly, the present invention is directed to suppressing a decrease in yield of a (poly)alkylene glycol monoalkyl ether per hour when an olefin is reacted with a (poly)alkylene glycol in the presence of a catalyst to produce the (poly)alkylene glycol monoalkyl ether over a long period of time.

As a result of intensive studies to solve the above issue, the present inventors have focused attention on a branched olefin which is contained in a trace amount in an olefin raw material to be used and is by-produced during the reaction. An equilibrium yield of the (poly)alkylene glycol monoalkyl ether to be produced by the reaction of the branched olefin contained in the reaction raw materials is lower than that of a linear olefin, and thus, in a case where unreacted raw materials are recycled, an unreacted branched olefin accumulates in the reaction system over time.

When the concentration of the branched olefin is high, the yield of the (poly)alkylene glycol monoalkyl ether is lowered, the productivity is lowered, and the catalytic activity is lowered. Thus, the present inventors have found a method for stably producing a (poly)alkylene glycol monoalkyl ether over a long period of time by at least partially removing the branched olefin in the raw materials to be recovered and used by a method such as distillation to reduce the amount of the branched olefin in the reaction system to a specific amount or less, leading to completion of the present invention.

That is, the present invention encompasses the following.

1. A method for producing a (poly)alkylene glycol monoalkyl ether including reacting an olefin and a (poly)alkylene glycol in a reactor in the presence of a catalyst, the method including recovering at least a portion of raw materials used for the production and using the recovered raw materials again as a raw material, wherein at least one of the recovered raw materials contains an olefin, and a mass of a branched olefin relative to a mass sum of the branched olefin and a linear olefin contained in the recovered raw materials is controlled not to exceed 20 mass %.

2. The method according to 1, wherein the olefin is an olefin having 6 or more and 20 or less carbon atoms.

3. The method according to 1 or 2, wherein the recovered raw materials contain an olefin from which a branched olefin has been separated by distillation.

4. The method according to any one of 1 to 3, wherein at least one of the recovered raw materials contains a (poly)alkylene glycol.

5. The method according to any one of 1 to 4, wherein a solid acid catalyst is used as the catalyst.

6. The method according to 5, wherein a crystalline metallosilicate is used as the solid acid catalyst.

7. The method according to any one of 1. to 6., further including controlling the mass of the branched olefin relative to the mass sum of the branched olefin and the linear olefin contained in the recovered raw materials not to be 1.5 mass % or less.

The present invention also encompasses the following.

(1) A method for producing a (poly)alkylene glycol monoalkyl ether by reacting an olefin with a (poly)alkylene glycol in the presence of a catalyst, wherein when the (poly)alkylene glycol monoalkyl ether is synthesized and at least a portion of raw materials is recovered after the synthesis and used again as a raw material, the reaction is performed with a mass of a branched olefin in reaction raw materials being 1 mass % or more and 20 mass % or less relative to a mass sum of the branched olefin and a linear olefin contained in the reaction raw material.

(2) The production method according to (1), wherein the olefin is an olefin having 6 or more and 20 or less carbon atoms.

(3) The production method according to (1) or (2), wherein at least one of the recovered raw materials is an olefin.

(4) The production method according to (3), wherein the recovered raw material is an olefin and is a raw material prepared by separating the branched olefin by distillation.

(5) The production method according to any one of (1) to (4), wherein at least one of the recovered raw materials is a (poly)alkylene glycol.

(6) The production method according to any one of (1) to (5), wherein a solid acid catalyst is used as the catalyst.

(7) The production method according to (6), wherein a crystalline metallosilicate is used as the solid acid catalyst.

### BRIEF DESCRIPTION OF DRAWINGS

FIG. 1 illustrates an example of a reaction apparatus having a batch reactor.

FIG. 2 illustrates an example of a flow diagram of a reaction apparatus having a continuous tank-type reactor.

FIG. 3 illustrates an example of a flow diagram of the reaction apparatus having the continuous tank-type reactor.

### DESCRIPTION OF EMBODIMENTS

Hereinafter, embodiments of the present invention will be described. Note that the present invention is not limited to the following embodiments, and can be variously modified within the scope of the claims. The embodiments described in the present specification can be combined with each other in any manner to be another embodiment.

Throughout the present specification, it should be understood that the expression of a singular form includes the concept of a plural form thereof unless otherwise specified. Thus, it should be understood that a singular article (for example, “a”, “an”, “the”, and the like in the case of English) includes the concept of a plural form thereof unless otherwise specified. In addition, it should be understood that terms used in the present specification are used with the meanings commonly used in the art unless otherwise specified. Accordingly, unless defined otherwise, all technical and scientific terms used herein have the same meanings as commonly understood by those skilled in the art to which the present invention belongs. In case of conflict, the present specification (including definitions) will have priority.

In the present specification, “X to Y” indicating a range includes X and Y and means “X or more and Y or less”. Unless otherwise specified, operations and measurements of physical properties and the like are performed under conditions of room temperature (in a range of 20° C. or higher and 25° C. or lower)/relative humidity of 40% RH or more and 50% RH or less.

One aspect of the present invention is a method for producing a (poly)alkylene glycol monoalkyl ether including reacting an olefin and a (poly)alkylene glycol in a reactor in the presence of a catalyst, the method including recovering at least a portion of the raw materials used for the production and using the recovered raw materials again as a raw material, in which at least one of the recovered raw materials contains an olefin, and a mass of a branched olefin relative to a mass sum of the branched olefin and a linear olefin contained in the recovered raw materials is controlled not to exceed 20 mass %. With such a configuration, it is possible to produce the (poly)alkylene glycol monoalkyl ether in a high yield over a long period of time by reacting the olefin and the (poly)alkylene glycol in the presence of the catalyst.

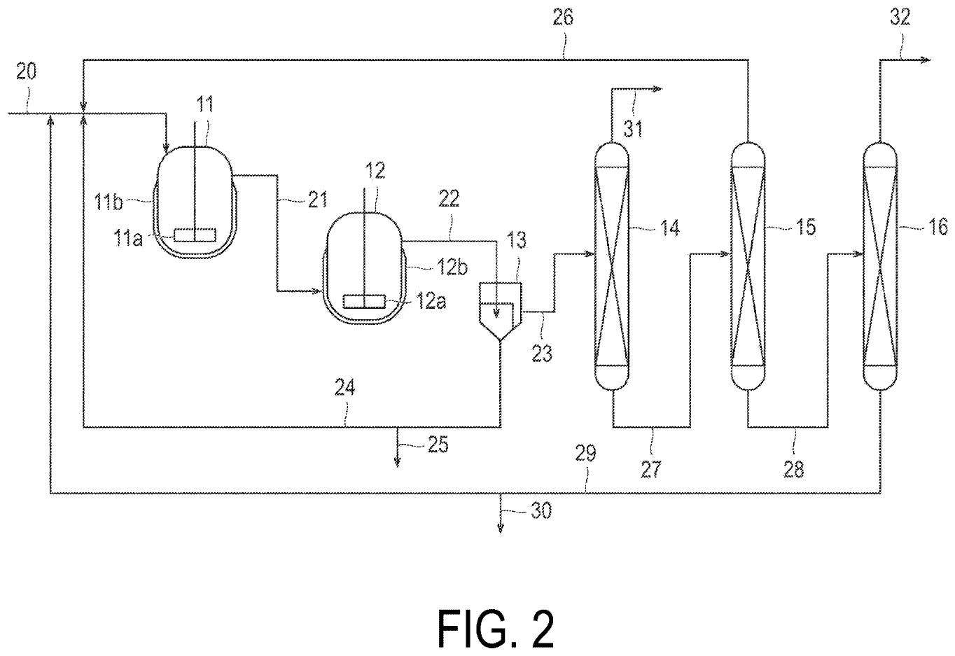

According to one embodiment of the present invention including reacting an olefin and a (poly)alkylene glycol in a reactor in the presence of a catalyst, a (poly)alkylene glycol monoalkyl ether is continuously produced. According to one embodiment of the present invention, a continuous operation time is 20 to 20000 hours, 100 to 15000 hours, or 200 to 10000 hours. The continuous operation time is preferably a time from when the recovered olefin treated to remove at least a portion of the branched olefin is led out from a facility (for example, a distillation column) in which the treatment is performed toward a reactor (in FIG. 2, when the recovered unreacted olefin is introduced into a conduit 26) to when supply of the raw materials to the reactor is stopped (in FIG. 2, when no raw material is supplied to a reactor 11).

The olefin to be used in the present invention is preferably a hydrocarbon having an ethylenically unsaturated bond and having 6 to 30 carbon atoms, more preferably a hydrocarbon having an ethylenically unsaturated bond and having 6 to 20 carbon atoms, and even more preferably a hydrocarbon having an ethylenically unsaturated bond and having 8 to 20 carbon atoms. The olefin to be used in the present invention is more preferably a hydrocarbon having an ethylenically unsaturated bond and 9 to 18 carbon atoms, or a hydrocarbon having an ethylenically unsaturated bond and 12 to 16 carbon atoms. In consideration of using the target (poly)alkylene glycol monoalkyl ether as a surfactant, the olefin preferably includes an acyclic olefin as a main component, and more preferably a linear olefin as a main component. The wording “acyclic (linear) olefin as a main component” means that the olefin contains the acyclic (linear) olefin in an amount of 80 mass % or more, 85 mass % or more, or 90 mass % or more. Here, a linear olefin which is industrially easily available usually contains a branched olefin, but it is preferable to use such a linear olefin as a raw material because it is inexpensive. These industrially easily available linear olefins contain a branched olefin in an amount ranging from several ppm to at most about several % although the concentration varies depending on a manufacturer of a raw material. The raw material olefin (fresh olefin) to be used in the production of the present invention is a linear olefin containing a branched olefin in an amount of preferably 0.01 to 10 mass %, more preferably 0.1 to 10 mass %, and even more preferably 1 to 8 mass %. The raw material olefin (fresh olefin) to be used in the present invention is a linear olefin containing a branched olefin in an amount of 1.2 to 7 mass %, 1.4 to 6 mass %, or 1.6 to 5.5 mass %.

Thus, the acyclic (linear) olefin (fresh olefin) that can be used as a raw material for the production of the (poly)alkylene glycol monoalkyl ether may be in the form of a mixture containing a branched olefin. However, such an olefin is not prepared to intentionally contain a branched olefin, and thus, even if it contains a branched olefin, it is also simply referred to as a linear olefin (fresh olefin, simply olefin). According to one embodiment of the present invention, the olefin contains no cyclic olefin, or contains a cyclic olefin in an amount of 1 mass % or less, 0.5 mass % or less, or 0.1 mass % or less.

Examples of the linear olefin include octene, nonene, decene, undecene, dodecene, tridecene, tetradecene, pentadecene, hexadecene, heptadecene, octadecene, nonadecene, eicosene, docosene, tricosene, and tetracosene. The number of carbon atoms of the linear olefin may be 6 to 30, 6 to 20, 8 to 20, 9 to 18, 10 to 17, or 12 to 16. These olefins can be used without particular limitation whether the position of an unsaturated bond is an α-position, an inner position, or a mixture of the α-position and the inner position.

In one embodiment of the present invention, the olefin has the unsaturated bond of the α-position (e.g., 1-dodecene, 1-tridecene, 1-tetradecene, 1-pentadecene, 1-hexadecene). In one embodiment of the present invention, the olefin has an unsaturated bond at an inner position (e.g., an inner dodecene, an inner tridecene, an inner tetradecene, an inner pentadecene, an inner hexadecene). Furthermore, two or more types of olefins having different carbon numbers may be mixed and used as a raw material. In one embodiment of the present invention, the olefin is a mixture of an olefin having an unsaturated bond at the α-position and an olefin having an unsaturated bond at an inner position (e.g., a mixture of 1-dodecene and an inner dodecene, a mixture of 1-tridecene and an inner tridecene, a mixture of 1-tetradecene and an inner tetradecene, a mixture of 1-pentadecene and an inner pentadecene, a mixture of 1-hexadecene and an inner hexadecene). In the reaction process of the present invention, a reaction in which isomerization in the position of the unsaturated bond in the olefin occurs simultaneously. Generally, in a case of a linear olefin, an inner olefin is thermodynamically more stable than an α-olefin. Thus, in a case where an α-olefin is used as a raw material, the olefin is gradually isomerized to an inner olefin during the reaction. The rate of isomerization varies depending on a reaction temperature and a type and an amount of a catalyst. Generally, in a case of a branched olefin, a structure in which many olefin sites are substituted is thermodynamically stable. Thus, in a case where a branched α-olefin is used, the olefin is gradually isomerized to an inner olefin having a larger number of substituents during the reaction. For example, 2-methyl-1-alkene with a methyl substituent at position 2, which is contained in a relatively large amount in an industrially easily available linear α-olefin, is gradually isomerized to 2-methyl-2-alkene during the reaction. The rate of isomerization varies depending on a reaction temperature and a type and an amount of a catalyst.

Examples of the (poly)alkylene glycol to be used in the production of the (poly)alkylene glycol monoalkyl ether in the present invention include monoethylene glycol, diethylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, and 1,4-cyclohexanemethanediol. These may be used alone or as a mixture of two or more thereof. Among these, monoethylene glycol, diethylene glycol, and triethylene glycol are preferable, and monoethylene glycol is more preferable. According to one embodiment of the present invention, the number of carbon atoms of alkylene in the (poly)alkylene glycol is 1 to 8, 1 to 6, 1 to 4, or 1 to 3, and most preferably 2.

As the catalyst to be used in the present invention, an acidic catalyst is suitable. Examples thereof include homogeneous catalysts such as sulfuric acid, benzenesulfonic acid, dodecylbenzenesulfonic acid, and heteropoly acids (phosphotungstic acid, phosphomolybdic acid, silicotungstic acid, and silicomolybdic acid); acidic ion exchange resins; composite metal oxides such as silica-alumina and titania-silica; and solid acid catalysts such as zeolite. These catalysts may be used alone or in combination of two or more thereof. Among them, a solid acid catalyst is preferable as the catalyst. The solid acid catalyst can be used repeatedly and continuously as compared with a homogeneous catalyst, and thus it is particularly effective when used in a long-term reaction as in the present invention.

Among these, a crystalline metallosilicate is particularly preferred. The crystalline metallosilicate is an ordered porous material having a constant crystal structure. That is, this is a solid material having a large number of ordered voids or pores in its structure and a large specific surface area. The crystalline metallosilicate to be used in the present invention is a crystalline aluminosilicate (also generally referred to as zeolite) or a compound in which another metal element is introduced into the crystal lattice instead of an Al atom of the crystalline aluminosilicate. Specific examples of such another metal element include B, Ga, In, Ge, Sn, P, As, Sb, Sc, Y, La, Ti, Zr, V, Cr, Mn, Fe, Co, Ni, Cu, and Zn, and these may be used alone or in a mixture of two or more thereof. A crystalline aluminosilicate, a crystalline ferrosilicate, a crystalline borosilicate, and a crystalline gallosilicate are preferred from the viewpoint of catalytic activity and ease of synthesis and availability, and a crystalline aluminosilicate is particularly preferred.

According to one embodiment of the present invention, the specific surface area of the catalyst is from 150 to 1500 m²/g, or from 300 to 1000 m²/g.

Specific examples of the crystalline metallosilicate to be used in the present invention include those having structures such as MFI (e.g., ZSM-5), MEL (e.g., ZSM-11), BEA (e.g., β-type zeolite), FAU (e.g., Y-type zeolite), MOR (e.g., mordenite), MTW (e.g., ZSM-12), and LTL (e.g., L-type zeolite) when described using the IUPAC code named by the Structure Commission of the International Zeolite Association. In addition to these, those having structures described in “ZEOLITES, Vol. 12, No. 5, 1992” and “HANDBOOK OF MOLECULAR SIEVES, written by R. Szostak, published by VAN NOSTRAND REINHOLD” can be exemplified. These may be used alone or in combination of two or more thereof. Among these, those having a structure of BEA are particularly preferable from the viewpoint of excellent catalytic activity.

The crystalline metallosilicate to be used in the present invention preferably has an atomic ratio of silicon atoms to metal atoms constituting the crystalline metallosilicate of 5 or more and 1500 or less, and particularly 10 or more and 500 or less. When the atomic ratio of silicon atoms to metal atoms is too small or too large, the catalytic activity is low, which is not preferable. Such a crystalline metallosilicate has ion-exchangeable cations outside the crystal lattice, and specific examples of these cations include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, and R₄P⁺ in which R is H or an alkyl group. Among them, those in which all or a part of cations are substituted with hydrogen ions (H⁺) are preferable as the catalyst of the present invention.

The crystalline metallosilicate to be used in the present invention can be synthesized by a generally used synthesis method, for example, a hydrothermal synthesis method. Such a crystalline metallosilicate can be synthesized, for example, by heating a composition containing a silica source, a metal source, and a quaternary ammonium salt such as a tetraethylammonium salt or a tetrapropylammonium salt at a temperature of about 100 to 175° C. until crystals are formed, followed by filtration, washing with water, and drying of a solid product, and then firing the dried solid product at 350 to 600° C. Metallosilicates having different crystal systems can be prepared by appropriately arranging raw materials and synthesis conditions.

As the silica source, water glass, silica sol, silica gel, alkoxysilane, and the like can be used. As the metal source, various inorganic or organic metal compounds can be used. Suitable examples of the metal compounds include metal salts such as metal sulfates [e.g., Al₂(SO₄)₃], metal nitrates [e.g., Fe(NO₃)₃], alkali metal salts of metal oxides [e.g., NaAlO₂]; metal halides such as metal chlorides [e.g., TiCl₄], and metal bromides [e.g., MgBr₂]; and metal alkoxides [e.g., Ti(OC₂H₅)₄]. As necessary, the prepared crystalline metallosilicate can be ion-exchanged to a desired cationic form. For example, an H⁺-type cationic form can be prepared by mixing and stirring the crystalline metallosilicate in an aqueous solution of HCl, NH₄Cl, NH₃, or the like to exchange the cationic species to an H⁺-type or NH₄⁺-type, followed by filtration, washing with water, and drying of a solid product, and then firing the dried product at 350 to 600° C. A cationic form other than H⁺ can be prepared by performing the same operation using an aqueous solution containing a desired cation.

As these crystalline metallosilicates, crystalline metallosilicates of a single crystal system may be used, or crystalline metallosilicates of various crystal systems may be used in combination. In the present invention, the catalyst may be used in any form, for example, in a form of powder, granules, or a molded body having a specific shape. In a case where a molded body is used, alumina, silica, titania, or the like may be used as a carrier or a binder. In a case where a homogeneous catalyst is used as the catalyst, it can be dissolved in a reaction raw material before use.

The reaction between the olefin and the (poly)alkylene glycol in the present invention can be performed either in the presence or absence of a solvent. As the solvent, a solvent such as nitromethane, nitroethane, nitrobenzene, dioxane, ethylene glycol dimethyl ether, diglyme, sulfolane, benzene, toluene, xylene, hexane, cyclohexane, decane, or paraffin can be used.

The reaction between the olefin and the (poly)alkylene glycol in the present invention can be performed by a generally used method such as a batch reaction or a flow reaction, and is not particularly limited. A molar ratio between the olefin and the (poly)alkylene glycol, which are raw materials for the reaction, is not particularly limited, but the molar ratio of the (poly)alkylene glycol to the olefin is preferably 0.05 to 20, more preferably 0.1 to 10, and even more preferably 1 to 5. A reaction temperature is preferably 50 to 250° C., and more preferably 100 to 200° C. A reaction pressure may be any of reduced pressure, ordinary pressure, and increased pressure, but is preferably in a range from ordinary pressure to 2 MPa.

According to one embodiment of the present invention, the mass of the catalyst relative to the mass of the (poly)alkylene glycol is suitably 0.1 to 100 mass %, 0.5 to 50 mass %, or 1 to 20 mass %.

According to one embodiment of the present invention, there is provided a method for producing a (poly)alkylene glycol monoalkyl ether including reacting an olefin and a (poly)alkylene glycol in a reactor in the presence of a catalyst, the method including recovering at least a portion of raw materials used for the production and using the recovered raw materials again as a raw material, in which at least one of the recovered raw materials contains an olefin, and a molar ratio of the (poly)alkylene glycol to the olefin (branched olefin and linear olefin) supplied to the reactor is controlled to be preferably 0.05 to 20, more preferably 0.1 to 10, and even more preferably 1 to 5.

According to one embodiment of the present invention, there is provided a method for producing a (poly)alkylene glycol monoalkyl ether including reacting an olefin and a (poly)alkylene glycol in a reactor in the presence of a catalyst, the method including recovering at least a portion of raw materials used for the production and using the recovered raw materials again as a raw material, in which at least one of the recovered raw materials contains an olefin, and a mass of the catalyst relative to a mass of the (poly)alkylene glycol supplied to the reactor is controlled to be 0.1 to 100 mass %, 0.5 to 50 mass %, or 1 to 20 mass %.

In the reaction of the olefin with the (poly)alkylene glycol, a branched olefin, a (poly)alkylene glycol dialkyl ether, and an alcohol are generated by side reactions. The branched olefin is generated during the reaction by an acid-catalyzed isomerization reaction of a linear olefin. The branched olefin is also generated by a reverse reaction due to an acid reaction from the product or the (poly)alkylene glycol dialkyl ether. While the branched olefin is generated by these side reactions, a yield of the side reaction from the linear olefin to the branched olefin is not high, and there is no particular problem in a short-term reaction. In a case where the process of recovering and recycling an unreacted raw material is performed for a long period of time, accumulation is particularly observed in the reaction system.

The (poly)alkylene glycol dialkyl ether and/or alcohol may be supplied to the reaction system of the olefin and the (poly)alkylene glycol because the (poly)alkylene glycol alkyl ether can be selectively prepared, and the supply amount thereof is not particularly limited. The (poly)alkylene glycol dialkyl ether or alcohol generated as a by-product may be recovered and accumulated and then supplied to the reaction system at once, or the (poly)alkylene glycol dialkyl ether or alcohol generated as a by-product in the previous reaction may be always supplied to the next reaction. When the (poly)alkylene glycol monoalkyl ether is continuously produced by performing the flow-type reaction, it is preferable that the (poly)alkylene glycol dialkyl ether and the alcohol generated as by-products be continuously recovered and always recycled and supplied to the reaction system. The generation amount of the (poly)alkylene glycol dialkyl ether or alcohol generated as a by-product by the reaction between the olefin and the (poly)alkylene glycol varies depending on types and a molar ratio of the olefin and the (poly)alkylene glycol, a type of the catalyst used, the reaction temperature, the reaction time, and the like, but is usually in a range from 0.0001 to 30 mol % relative to the olefin as a raw material. Alternatively, depending on the type of the catalyst used, the types of the raw materials, the reaction conditions, and the like, the (poly)alkylene glycol dialkyl ether or the alcohol may not be substantially generated as a by-product. In some cases, the (poly)alkylene glycol dialkyl ether or the alcohol generated as by-products is recovered as a product. In such a case, the (poly)alkylene glycol dialkyl ether or the alcohol only need be supplied to the reaction system of the olefin and the (poly)alkylene glycol.

In a case where a batch reactor is used, a catalyst, and an olefin and a (poly)alkylene glycol as raw materials are put into the reactor, and as necessary, a (poly)alkylene glycol dialkyl ether and/or an alcohol is put into the reactor, followed by stirring at a predetermined temperature and a predetermined pressure to produce a mixture containing a (poly)alkylene glycol monoalkyl ether as a target product. The amount of the catalyst to be used is not particularly limited, but is preferably 0.1 to 100 mass %, more preferably 0.5 to 50 mass %, and even more preferably 1 to 20 mass % relative to the olefin as a raw material. The reaction time varies depending on the reaction temperature, the amount of the catalyst, a raw material composition ratio, and the like, but is in a range from 0.1 to 100 hours, and preferably 0.5 to 30 hours.

In a case where a flow-type reactor is used, the reaction can be performed by any of fluidized-bed type, moving-bed type, fixed-bed type, and stirred-tank type. The reaction conditions vary depending the raw material composition, a catalyst concentration, the reaction temperature, and the like, but a liquid hourly space velocity (LHSV), that is, a value obtained by dividing a volumetric flow rate of the flowing raw materials by a reactor volume, is preferably in a range from 0.01 to 50 hr⁻¹, and particularly from 0.1 to 20 hr⁻¹. The present invention is based on a long-term reaction, and thus it is preferable to use a flow-type reactor.

In a case where a fluidized-bed type, a moving-bed type, or a stirred-tank type reactor is used, it is preferable not to add a solvent, and the (poly)alkylene glycol and the olefin, which are raw materials, are only slightly soluble in each other, and only that amount is dissolved, and thus the reaction liquid is usually separated into two phases. The catalyst (solid catalyst such as crystalline metallosilicate) is dispersed and contained in a (poly)alkylene glycol phase, and the (poly)alkylene glycol monoalkyl ether as a product and the branched olefin, (poly)alkylene glycol dialkyl ether, and alcohol generated by the side reactions are mainly contained in an olefin phase. Accordingly, after completion of the reaction, the (poly)alkylene glycol phase and the olefin phase are separated from each other, and the target (poly)alkylene glycol monoalkyl ether can be prepared from the olefin phase by a method such as distillation or extraction.

On the other hand, in a case where a fixed-bed reactor is used, it is preferable to add a solvent during the reaction to compatibilize the (poly)alkylene glycol and the olefin, which are raw materials, under the reaction conditions. In this case, it is preferable that the (poly)alkylene glycol phase can be separated from the olefin phase containing the (poly)alkylene glycol monoalkyl ether as a product and the branched olefin, the (poly)alkylene glycol dialkyl ether, and the alcohol, which are generated by a side reaction because it is possible to reduce a cost required for separation after completion of the reaction. Specifically, the (poly)alkylene glycol phase and the olefin phase are separated after completion of the reaction by appropriately controlling the amount of the solvent to be added, the type of the solvent, and the temperature, or removing the solvent by a method such as distillation after completion of the reaction to perform phase separation, and the target (poly)alkylene glycol monoalkyl ether can be prepared from the olefin phase by a method such as distillation or extraction.

In addition, it is preferable that an olefin which has been used as a raw material but has been unreacted be recovered and can be used again as a raw material for the reaction with the (poly)alkylene glycol, and an olefin which is insufficient for the reaction is added. At this time, at least a portion of the branched olefin contained in the unreacted olefin is removed by distillation, and the remaining olefin from which at least a portion of the branched olefin has been removed can be supplied to the reaction system of the olefin and the (poly)alkylene glycol and used again as a raw material as described above.

Among the unreacted olefin, the (poly)alkylene glycol monoalkyl ether as the target product, and the branched olefin, the alcohol, and the (poly)alkylene glycol dialkyl ether, which are by-products, contained in the olefin phase, the branched olefin generally has the lowest boiling point, and the boiling point increases in the order of the linear olefin, the alcohol, the (poly)alkylene glycol monoalkyl ether, and the (poly)alkylene glycol dialkyl ether. Accordingly, it is also possible to first remove the branched olefin as a fraction by distillation, then recover the unreacted olefin and the alcohol as a fraction, then recover (prepare) the (poly)alkylene glycol monoalkyl ether as a product, and recover the (poly)alkylene glycol dialkyl ether as a distillation bottom. Further, the (poly)alkylene glycol monoalkyl ether recovered as a product can be further purified by distillation or washing. The unreacted olefin from which at least a portion of the branched olefin has been removed and the alcohol and/or the (poly)alkylene glycol dialkyl ether as a by-product can be recycled to be used in the reaction system of the olefin and the (poly)alkylene glycol. In addition, for the purpose of purging impurities such as a heavy component, a portion of the distillation bottom can be discarded and the remainder can be supplied to the reaction system of the olefin and the (poly)alkylene glycol and recycled to be used.

The boiling points of the linear olefin and the branched olefin are close to each other, and thus when the branched olefin is distilled off from the unreacted olefin, the linear olefin is distilled off together with the branched olefin. Thus, when all the branched olefin is distilled off, the recovery rate of the unreacted olefin decreases, and utilization efficiency of the olefin decreases in a long-term reaction (that is, an amount of the olefin to be removed increases relative to the amount of the olefin put). Here, the utilization efficiency of the olefin is obtained from the following equation.

Utilization efficiency of olefin=(total number of moles of finally produced (poly)alkylene glycol monoalkyl ether)/(total number of moles of fresh olefin put as a raw material).

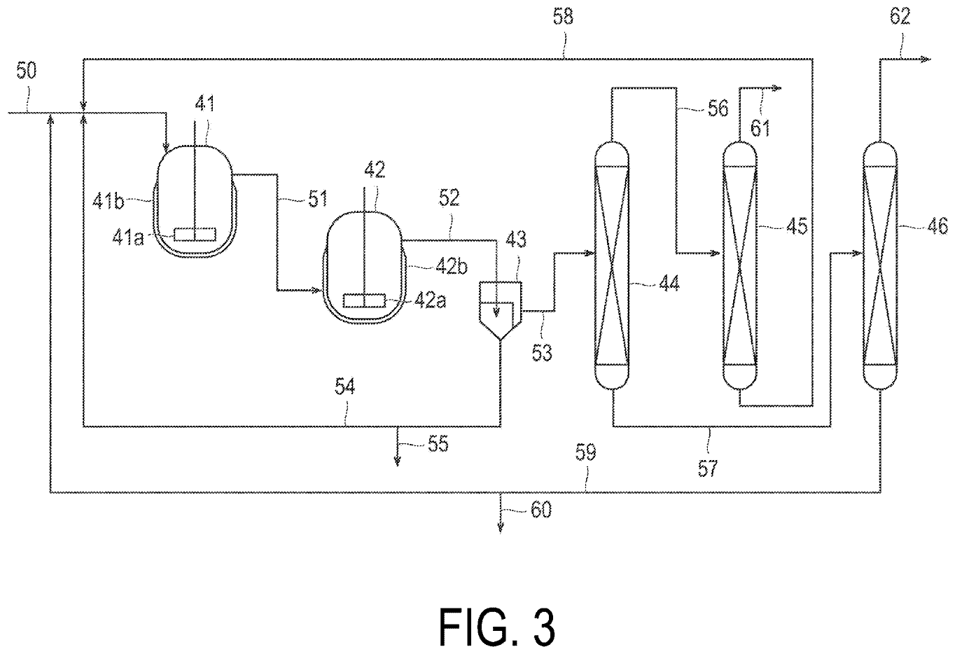

For such a reason, when the unreacted olefin is recycled to be used, it is preferable not to distill off all of the branched olefin from the unreacted olefin. Specifically, it is preferable to control the concentration of the branched olefin in the olefin (branched olefin and linear olefin flowing through the conduit 26 in FIG. 2, branched olefin and linear olefin flowing through a conduit 58 in FIG. 3) to be recovered and used again as a raw material to be a certain value or more. Specifically, the mass of the branched olefin relative to the mass sum of the olefins to be recovered and used again as a raw material (including branched olefin and linear olefin) is controlled not to be less than 1 mass %. In the present specification, the mass of the branched olefin relative to the mass sum of the branched olefin and the linear olefin is also referred to as a branched olefin ratio.

As described above, when the branched olefin ratio is high, the yield of the (poly)alkylene glycol monoalkyl ether decreases and the catalytic activity decreases. Thus, the branched olefin ratio in the olefins to be recovered and used again as a raw material (branched olefin and linear olefin flowing through the conduit 26 in FIG. 2, branched olefin and linear olefin flowing through the conduit 58 in FIG. 3) is controlled not to exceed 20 mass %.

When the branched olefin ratio is controlled to be in such a range, a decrease in the yield of the (poly)alkylene glycol monoalkyl ether due to the branched olefin can be suppressed, and the utilization efficiency of the olefin can be maintained at a high level (that is, the amount of the olefin to be removed relative to the amount of the olefin put can be reduced). According to one embodiment of the present invention, the mass of the branched olefin (branched olefin ratio) relative to the mass sum of the branched olefin and the linear olefin present in the reaction raw materials (olefins) to be recovered and used again as a raw material is controlled not to be 1.5 mass % or less, 1.7 mass % or less, 2 mass % or less, 2.2 mass % or less, 2.4 mass % or less, 2.6 mass % or less, or 2.8 mass % or less. According to one embodiment of the present invention, the mass of the branched olefin (branched olefin ratio) relative to the mass sum of the branched olefin and the linear olefin present in the reaction raw materials (olefins) to be recovered and used again as a raw material is controlled not to be more than 20 mass %, 15 mass % or more, 10 mass % or more, 9 mass % or more, 8 mass % or more, 7 mass % or more, or 6 mass % or more. Note that the branched olefin ratio is measured as follows. That is, it is calculated from the area of the linear olefin and the area of the branched olefin measured using gas chromatography (GC) in accordance with the following equation.

Branched olefin ratio=(area obtained from GC analysis of linear olefin)/{(area obtained from GC analysis of linear olefin)+(area obtained from GC analysis of branched olefin)}.

Note that analysis conditions by gas chromatography (GC) are as follows:

### GC Conditions

Gas chromatography apparatus: available from Shimadzu Corporation, product name: Nexis (trade name) GC-2030
Column: available from Agilent Technologies, Inc., product name: DB-1
(column length: 60 m, inner diameter: 0.25 mm, film thickness: 0.25 μm)
Detector: FID detection method
Injection volume: 1 μL
Linear velocity: 14 cm/sec
Carrier gas: N₂

Temperature raising condition: temperature raising from 60° C. to 100° C. at 3° C./min, then temperature raising to 320° C. at 10° C./min, and holding at 320° C. for 30 minutes.

In the present invention, the mass of the branched olefin (branched olefin ratio) relative to the mass sum of the branched olefin and the linear olefin contained in the raw materials supplied to the reactor is also preferably controlled not to exceed 20 mass %, more preferably not to be 15 mass % or more, 10 mass % or more, 9 mass % or more, 8 mass % or more, 7 mass % or more, or 6 mass % or more. In addition, the mass of the branched olefin relative to the mass sum of the branched olefin and the linear olefin contained in the raw materials to be supplied to the reactor is also preferably controlled not to be less than 1 mass %, and more preferably controlled not to be 1.5 mass % or less, 1.7 mass % or less, 1.9 mass % or less, 2 mass % or less, 2.2 mass % or less, 2.4 mass % or less, 2.6 mass % or less, or 2.8 mass % or less.

In a case where a fluidized-bed type, moving-bed type, or stirred-tank type reactor is used, the catalyst can be separated from the (poly)alkylene glycol phase containing the catalyst by a method such as centrifugal separation, filtration, or drying and recycled to be used for the next reaction. In addition, the (poly)alkylene glycol can be recovered from the (poly)alkylene glycol phase by a method such as distillation, recycled for the next reaction to be used for the reaction with the olefin. The (poly)alkylene glycol phase containing the catalyst is recycled for the next reaction to be used for the reaction with the olefin, and at this time, it is preferable to perform the reaction after (or while) replenishing the (poly)alkylene glycol consumed by the reaction by that amount because the process and the like become simple. In this case, the activity of the catalyst may be gradually decreased by the reaction, and thus, in a case where it is recognized that the activity of the catalyst is decreased, at least a portion of the catalyst can be extracted and regenerated, or newly replenished to be supplied to the next reaction. In a case where impurities such as a heavy component accumulate in the (poly)alkylene glycol phase, a portion of the (poly)alkylene glycol phase may be extracted for the purpose of purging these impurities, and the remainder may be recycled in the next reaction. On the other hand, in a case where a fixed-bed reactor is used and the activity of the catalyst is lowered by the reaction, the catalyst may be regenerated in the fixed bed or replaced to improve the activity. In a case where a fixed-bed reactor is used, it is preferable to prepare at least two reactors and alternately perform the reaction and the catalyst regeneration because it is not necessary to stop the reaction when the catalyst is regenerated.

A distillation temperature depends on a material to be separated by distillation. For example, the temperature at the top of a distillation column is usually 15 to 300° C., or 50 to 300° C., preferably 60 to 300° C., and more preferably 70 to 280° C.

A distillation retention time is usually within 24 hours, preferably within 12 hours, and more preferably within 6 hours. The distillation retention time is preferably 5 minutes or longer, more preferably 10 minutes or longer, and even more preferably 15 minutes or longer. The distillation may be performed under ordinary pressure or reduced pressure, but is preferably under reduced pressure, and the degree of reduced pressure is preferably 15 kPa or less, and more preferably 10 kPa or less. The degree of reduced pressure is preferably 50 Pa or more, and more preferably 100 Pa or more. The number of theoretical stages of the distillation column is preferably 2 or more, more preferably 3 or more, and even more preferably 5 or more, although it depends on that to be separated by distillation. When the number of stages is small, separation by distillation may not be performed. The number of theoretical stages is preferably 150 stages or less, more preferably 100 stages or less, and even more preferably 50 stages or less. When the number of theoretical stages is increased, the distillation column becomes large and a fixed cost increases, which is not preferable from an industrial viewpoint.

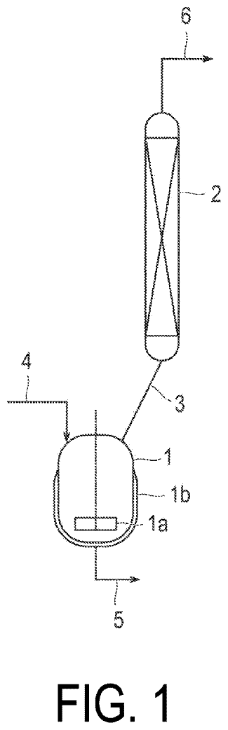

Next, an embodiment of the present invention will be described with reference to the drawings. First, an example of a method for producing a (poly)alkylene glycol monoalkyl ether using a reaction apparatus having a batch reactor as a reactor will be described with reference to FIG. 1. As illustrated in FIG. 1, the reaction apparatus includes a batch reactor 1 and a distillation column 2. The batch reactor 1 is pressure-resistant and includes a stirring device 1a and a heating device 1b. A raw material supply pipe 4 and an extraction pipe 5 are connected to the batch reactor 1. The upper portion of the batch reactor 1 and the bottom of the distillation column 2 are connected by a conduit 3, and a gas generated from the batch reactor 1 can be introduced into the distillation column 2 and a column bottom liquid of the distillation column 2 can be returned to the batch reactor 1. An extraction pipe 6 for extracting a distillate fraction is connected to the top of the distillation column 2.

First, a first reaction is performed in the absence of a (poly)alkylene glycol dialkyl ether and/or an alcohol. An olefin, a (poly)alkylene glycol, a catalyst, and optionally a solvent, which are reaction raw materials, are put into the batch reactor 1 through the raw material supply pipe 4. Next, the reaction liquid is heated with being stirred and is reacted under predetermined temperature and pressure conditions to synthesize a (poly)alkylene glycol monoalkyl ether. At this time, a branched olefin, a (poly)alkylene glycol dialkyl ether and/or an alcohol are generated as by-products. After completion of the reaction, the stirrer is stopped, and the mixture is allowed to stand to separate into a catalyst, a (poly)alkylene glycol phase (lower layer) and an olefin phase (upper layer) containing a (poly)alkylene glycol monoalkyl ether and a branched olefin as a by-product. At this time, the catalyst may be dispersed in the (poly)alkylene glycol phase depending on the shape and size of the catalyst.

Note that in a case where the reaction liquid is not separated after the reaction because a solvent is used in the reaction, the temperature of phase separation may be changed to a temperature at which the reaction liquid is separated into two phases, or the solvent may be first removed by distillation or the like to perform phase separation, but it is preferable to first separate the catalyst. When the catalyst is recoverable by filtration or the like, it is recovered. In a case where the catalyst cannot be separated depending on the shape, size, and the like of the catalyst, it is preferable to separate a (poly)alkylene glycol phase (lower layer) containing the catalyst and an olefin phase (upper layer) containing a (poly)alkylene glycol monoalkyl ether and a branched olefin as a by-product.

Thereafter, the separated catalyst or the (poly)alkylene glycol phase containing the catalyst is extracted from the batch reactor 1 through the extraction pipe 5. The olefin phase remaining in the batch reactor 1 is separated into individual components by batch distillation. Under controlling pressures of the batch reactor 1 and the distillation column 2, a temperature of the olefin phase remaining in the batch reactor 1, and a reflux ratio of the distillation column 2, the components present in the olefin phase are taken out as a distillate from the top of the distillation column through the extraction pipe 6 in the order from the component having the lowest boiling point. First, at least a portion of the branched olefin as a by-product is removed, then an unreacted olefin from which at least a portion of the branched olefin has been removed and an alcohol as a by-product are recovered, and then a (poly)alkylene glycol monoalkyl ether as a target product is recovered. Note that the branched olefin, the unreacted olefin, and the alcohol as a by-product may be recovered at the same time in advance, and a branched olefin as a by-product may be removed from this liquid in another distillation column. The (poly)alkylene glycol dialkyl ether as a by-product may be subsequently recovered by distillation, or may be left in the batch reactor 1 as a distillation bottom and supplied to the next batch reaction. Note that the distillation of the olefin phase can also be performed using a distillation device (not illustrated) other than the distillation column 2.

Next, the second and subsequent reactions will be described. In the second and subsequent reactions, the (poly)alkylene glycol dialkyl ether and/or alcohol generated as a by-product is supplied to the reaction system to perform the reaction. In addition, the unreacted olefin from which at least a portion of the branched olefin has been removed and the (poly)alkylene glycol phase are also reused for the reaction. The unreacted olefin recovered by the previous batch reaction from which at least a portion of the branched olefin has been removed, the (poly)alkylene glycol phase containing the catalyst, and the (poly)alkylene glycol dialkyl ether and/or alcohol generated as a by-product are used as reaction raw materials, and the olefin and (poly)alkylene glycol consumed by the previous reaction are replenished and put into the batch reactor 1 through the raw material supply pipe 4. Note that in a case where the (poly)alkylene glycol dialkyl ether is left in the batch reactor 1 as a distillation bottom, it is not necessary to supply the (poly)alkylene glycol dialkyl ether through the raw material supply pipe 4. After the raw materials are supplied, the reaction is performed under the same conditions as in the previous reaction, and each component is separated and recovered under the same conditions as in the previous reaction. When the batch reaction is repeated while the branched olefin which is the lightest boiling component is removed during such distillation, the branched olefin which is a by-product does not accumulate in the system, and the (poly)alkylene glycol dialkyl ether and/or alcohol which are by-products are converted into the (poly)alkylene glycol monoalkyl ether, whereby the (poly)alkylene glycol monoalkyl ether can be produced from the olefin and the (poly)alkylene glycol with high selectivity and high efficiency. Note that in a case where impurities such as a heavy component are accumulated in the (poly)alkylene glycol phase and the olefin phase by repeating the batch reaction, the heavy component can be removed by purging a portion of the (poly)alkylene glycol phase or purging a portion of the bottom produced by distilling the olefin phase.

Next, an example of a method for producing a (poly)alkylene glycol monoalkyl ether using a reaction apparatus having a flow-type reactor as a reactor will be described with reference to FIGS. 2 and 3.

The reaction apparatus may be any one of a fluidized-bed type, a moving-bed type, a fixed-bed type and a stirred-tank type, but here, as illustrated in FIG. 2 and FIG. 3, a case of using a continuous tank-type reactor is used as an example.

In FIG. 2, the reaction apparatus having a flow-type reactor includes continuous tank-type reactors 11 and 12 and distillation columns 14, 15, and 16. The continuous tank-type reactor 11 includes a stirring device 11a and a heating device 11b. The continuous tank-type reactors 12 includes a stirring device 12a and a heating device 12b. A raw material supply pipe 20 is connected to the continuous tank-type reactor 11, and an overflow-type conduit 21 is connected to an upper portion of the continuous tank-type reactor 11. The conduit 21 also serves as a raw material supply pipe for the continuous tank-type reactor 12. An overflow-type conduit 22 is connected to an upper portion of the continuous tank-type reactor 12 for introduction into a liquid-liquid separator (settler) 13. The liquid-liquid separator 13 and the distillation column 14 are connected by a conduit 23, and a liquid of the upper layer separated by the liquid-liquid separator 13 is introduced into the distillation column 14. The liquid-liquid separator 13 and the raw material supply pipe 20 are connected by a conduit 24, and a liquid of the lower layer separated by the liquid-liquid separator 13 can be returned to the continuous tank-type reactor 11. A conduit 25 is connected to the middle of the conduit 24. The bottom of the distillation column 14 and the distillation column 15 are connected by a conduit 27, and the column bottom liquid of the distillation column 14 is introduced into the distillation column 15. The top of the distillation column 14 is connected to a conduit 31. The bottom of the distillation column 15 and the distillation column 16 are connected by a conduit 28, and the column bottom liquid of the distillation column 15 is introduced into the distillation column 16. The top of the distillation column 15 and the raw material supply pipe 20 are connected by a conduit 26, and the distillate fraction from the top of the distillation column 15 can be returned to the continuous tank-type reactor 11. The bottom of the distillation column 16 and the raw material supply pipe 20 are connected by a conduit 29, and the column bottom liquid of the distillation column 16 can be returned to the continuous tank-type reactor 11. A conduit 30 is connected to the middle of the conduit 29. A conduit 32 is connected to the top of the distillation column 16.

First, a linear olefin, a (poly)alkylene glycol, a catalyst, and optionally a solvent, which are reaction raw materials, are continuously put into the continuous tank-type reactor 11 through the raw material supply pipe 20. Next, the reaction liquid is heated with being stirred and is reacted under predetermined temperature and pressure conditions to synthesize a (poly)alkylene glycol monoalkyl ether. At this time, a branched olefin and a (poly)alkylene glycol dialkyl ether and/or an alcohol are generated as by-products. An overflow portion of the reaction liquid is introduced into the continuous tank-type reactor 12 to continue the reaction, and the overflow portion is introduced into the liquid-liquid separator 13. In the liquid-liquid separator 13, the overflow portion is separated into a (poly)alkylene glycol phase (lower layer) containing the catalyst and an olefin phase (upper layer) containing the (poly)alkylene glycol monoalkyl ether, the branched olefin, the (poly)alkylene glycol dialkyl ether, and the alcohol. Thereafter, the (poly)alkylene glycol phase is extracted through the conduit 24 and put into the continuous tank-type reactor 11 through the raw material supply pipe 20, and at this time, the (poly)alkylene glycol is replenished as necessary in an amount corresponding to the amount consumed by the reaction. A portion of the (poly)alkylene glycol phase is extracted from the conduit 25 connected to the middle of the conduit 24 to regenerate a portion of the catalyst. The catalyst and the (poly)alkylene glycol are recovered from the (poly)alkylene glycol phase extracted from the conduit 25 to regenerate the catalyst. The regenerated catalyst and the recovered (poly)alkylene glycol are again supplied to the continuous tank-type reactor 11 through the raw material supply pipe 20. Note that in a case where impurities such as a heavy product generated by a side reaction such as dehydration condensation and water are accumulated in the (poly)alkylene glycol phase, they can be removed to the outside of the system during the extraction of a portion of the (poly)alkylene glycol phase for regeneration of the catalyst. The olefin phase of the upper layer in the liquid-liquid separator 13 is introduced into the distillation column 14 through the conduit 23. Under controlling the pressure in the distillation column 14, the temperature of the olefin phase, and the reflux ratio of the distillation column 14, a low-boiling point component present in the olefin phase, that is, the branched olefin, is at least partially removed through the conduit 31. This makes it possible to reduce the concentration of the branched olefin accumulated in the reaction system.

According to one embodiment of the present invention, a proportion of the (poly)alkylene glycol monoalkyl ether and the (poly)alkylene glycol dialkyl ether in the liquid introduced into the distillation column 14 (liquid flowing through the conduit 23) is usually more than 1.0 mass %.

According to one embodiment of the present invention, the column top pressure of the distillation column (distillation column 14 in FIG. 2) for removing at least a portion of the branched olefin is suitably 0.01 to 50 kPa, 0.05 to 20 kPa, or 0.1 to 10 kPa. According to one embodiment of the present invention, the column bottom temperature of the distillation column (distillation column 14 in FIG. 2) for removing at least a portion of the branched olefin is suitably 50 to 250° C., 70 to 220° C., or 80 to 200° C. According to one embodiment of the present invention, the column top temperature of the distillation column (distillation column 14 in FIG. 2) for removing at least a portion of the branched olefin is suitably 30 to 200° C., 50 to 180° C., or 60 to 150° C. According to one embodiment of the present invention, the reflux ratio of the distillation column (distillation column 14 in FIG. 2) for removing at least a portion of the branched olefin is suitably 0.01 to 300, 0.1 to 200, or 1 to 100.

The (unreacted) olefin from which the branched olefin has been at least partially removed, the (poly)alkylene glycol monoalkyl ether, the (poly)alkylene glycol dialkyl ether, and the alcohol are introduced into the distillation column 15 from the bottom of the distillation column 14 through the conduit 27.

According to one embodiment of the present invention, the branched olefin ratio in the liquid (to be introduced into the next distillation column) (liquid flowing through the conduit 27) treated to remove at least a portion of the branched olefin is controlled not to be less than 1 mass %, 1.5 mass % or less, 1.7 mass % or less, 1.9 mass % or less, 2 mass % or less, 2.2 mass % or less, 2.4 mass % or less, 2.6 mass % or less, or 2.8 mass % or less. According to one embodiment of the present invention, the branched olefin ratio in the liquid (to be introduced into the next distillation column) (liquid flowing through the conduit 27) treated to remove at least a portion of the branched olefin is controlled not to be more than 20 mass %, 15 mass % or more, 10 mass % or more, 9 mass % or more, 8 mass % or more, 7 mass % or more, or 6 mass % or more.

Under controlling the pressure of the distillation column 15, the temperature of the olefin phase, and the reflux ratio of the distillation column 15, components having the second lowest boiling point present in the olefin phase, that is, the unreacted olefin and the alcohol as a by-product are extracted as a distillate from the top of the distillation column 15 through the conduit 26.

According to one embodiment of the present invention, the branched olefin ratio in the conduit 26 is controlled not to be less than 1.0 mass %, preferably 1.5 mass % or less, 1.7 mass % or less, 1.9 mass % or less, 2 mass % or less, 2.2 mass % or less, 2.4 mass % or less, 2.6 mass % or less, or 2.8 mass % or less. According to one embodiment of the present invention, the branched olefin ratio in the conduit 26 is controlled not to be more than 20 mass %, 15 mass % or more, 10 mass % or more, 9 mass % or more, 8 mass % or more, 7 mass % or more, or 6 mass % or more.

According to one embodiment of the present invention, the column top pressure of the distillation column for recovering the unreacted olefin (distillation column 15 in FIG. 2) is suitably 0.01 to 50 kPa, 0.05 to 20 kPa, or 0.1 to 10 kPa.

According to one embodiment of the present invention, the column bottom temperature of the distillation column for recovering the unreacted olefin (distillation column 15 in FIG. 2) is suitably 100 to 300° C., 120 to 280° C., or 140 to 250° C. According to one embodiment of the present invention, the column top temperature of the distillation column for recovering the unreacted olefin (distillation column 15 in FIG. 2) is suitably 30 to 200° C., 50 to 180° C., or 70 to 150° C. According to one embodiment of the present invention, the reflux ratio of the distillation column for recovering the unreacted olefin (distillation column 15 in FIG. 2) is suitably 0.01 to 300, 0.05 to 100, or 0.1 to 50.

The olefin and the alcohol extracted from the top of the distillation column 15 are extracted through the conduit 26 and put into the continuous tank-type reactor 11 through the raw material supply pipe 20, and at this time, the linear olefin is replenished as necessary in an amount corresponding to the amount consumed by the reaction.

The (poly)alkylene glycol monoalkyl ether and the (poly)alkylene glycol dialkyl ether as a by-product, which have been extracted from the bottom of the distillation column 15, are introduced into the distillation column 16 through the conduit 28. Under controlling the pressure of the distillation column 16, the temperature of the (poly)alkylene glycol monoalkyl ether phase, and the reflux ratio of the distillation column 16, the (poly)alkylene glycol monoalkyl ether, which is the target reaction product as a component having a low boiling point, is extracted as a distillate from the top of the distillation column 16 through the conduit 32.

According to one embodiment of the present invention, the column top pressure of the distillation column (distillation column 16 in FIG. 2) for preparing the target (poly)alkylene glycol monoalkyl ether is suitably 10 to 3000 Pa, 20 to 1000 Pa, or 30 to 500 Pa. According to one embodiment of the present invention, the bottom temperature of the distillation column (distillation column 16 in FIG. 2) for preparing the target (poly)alkylene glycol monoalkyl ether is suitably 100 to 350° C., 130 to 320° C., or 150 to 280° C. According to one embodiment of the present invention, the column top temperature of the distillation column (distillation column 16 in FIG. 2) for preparing the target (poly)alkylene glycol monoalkyl ether is suitably 50 to 300° C., 80 to 280° C., or 100 to 250° C. According to one embodiment of the present invention, the reflux ratio of the distillation column (distillation column 16 in FIG. 2) for preparing the target (poly)alkylene glycol monoalkyl ether is suitably 0.01 to 300, 0.05 to 100, or 0.1 to 50.

The (poly)alkylene glycol dialkyl ether extracted from the bottom of the distillation column 16 is put into the continuous tank-type reactor 11 through the conduit 29 and further through the raw material supply pipe 20. In a case where impurities such as a heavy component accumulate in the (poly)alkylene glycol dialkyl ether phase, the heavy component can be removed by purging a portion of the (poly)alkylene glycol dialkyl ether phase through the conduit 30. When such a flow-type reaction is repeated, the (poly)alkylene glycol dialkyl ether and/or the alcohol as a by-product is converted into the (poly)alkylene glycol monoalkyl ether, whereby the (poly)alkylene glycol monoalkyl ether can be produced from the olefin and the (poly)alkylene glycol with high selectivity and high efficiency.

In FIG. 3, a reaction apparatus having a flow-type reactor includes continuous tank-type reactors 41 and 42 and distillation columns 44, 45, and 46. The continuous tank-type reactor 41 includes a stirring device 41a and a heating device 41b. The continuous tank-type reactor 42 includes a stirring device 42a and a heating device 42b. A raw material supply pipe 50 is connected to the continuous tank-type reactor 41, and an overflow-type conduit 51 is connected to an upper portion of the continuous tank-type reactor 41. The conduit 51 also serves as a raw material supply pipe for continuous tank-type reactor 42. An overflow-type conduit 52 is connected to an upper portion of the continuous tank-type reactor 42 for introduction into a liquid-liquid separator (settler) 43. The liquid-liquid separator 43 and the distillation column 44 are connected through a conduit 53, and a liquid of an upper layer separated by the liquid-liquid separator 43 is introduced into the distillation column 44. The liquid-liquid separator 43 and the raw material supply pipe 50 are connected through a conduit 54, and a liquid of a lower layer separated by the liquid-liquid separator 43 can be returned to the continuous tank-type reactor 41. A conduit 55 is connected to the middle of the conduit 54. The top of the distillation column 44 and the distillation column 45 are connected through a conduit 56, and a distillate of the distillation column 44 is introduced into the distillation column 45 through the conduit 56. The bottom of the distillation column 44 and the distillation column 46 are connected through a conduit 57, and a column bottom liquid of the distillation column 44 is introduced into the distillation column 46. The top of the distillation column 45 is connected to a conduit 61. The bottom of the distillation column 45 and the raw material supply pipe 50 are connected through a conduit 58, whereby a column bottom liquid of the distillation column 45 can be returned to the continuous tank-type reactor 11. The bottom of the distillation column 46 and the raw material supply pipe 50 are connected through a conduit 59, whereby a column bottom liquid of the distillation column 46 can be returned to the continuous tank-type reactor 41. A conduit 60 is connected to the middle of the conduit 59. A conduit 62 is connected to the top of the distillation column 46.

First, a linear olefin, a (poly)alkylene glycol, a catalyst, and optionally a solvent, which are reaction raw materials, are continuously put into the continuous tank-type reactor 41 through the raw material supply pipe 50. Next, the reaction liquid is heated with being stirred and is reacted under predetermined temperature and pressure conditions to synthesize a (poly)alkylene glycol monoalkyl ether. At this time, a branched olefin and a (poly)alkylene glycol dialkyl ether and/or an alcohol are generated as by-products. An overflow portion of the reaction liquid is introduced into the continuous tank-type reactor 42 to continue the reaction, and the overflow portion is introduced into the liquid-liquid separator 43. In the liquid-liquid separator 43, the overflow portion is separated into a (poly)alkylene glycol phase (lower layer) containing the catalyst and an olefin phase (upper layer) containing the branched olefin, the (poly)alkylene glycol monoalkyl ether, the (poly)alkylene glycol dialkyl ether, and the alcohol. Thereafter, the (poly)alkylene glycol phase is extracted through the conduit 54 and put into the continuous tank-type reactor 41 through the raw material supply pipe 50, and at this time, the (poly)alkylene glycol is replenished as necessary in an amount corresponding to the amount consumed by the reaction.

A portion of the (poly)alkylene glycol phase is extracted from the conduit 55 connected to the middle of the conduit 54 to regenerate a portion of the catalyst. The catalyst and the (poly)alkylene glycol are recovered from the (poly)alkylene glycol phase extracted from the conduit 55 to regenerate the catalyst. The regenerated catalyst and the recovered (poly)alkylene glycol are again supplied to the continuous tank-type reactor 41 through the raw material supply pipe 50. Note that in a case where impurities such as a heavy product generated by a side reaction such as dehydration condensation and water are accumulated in the (poly)alkylene glycol phase, they can be removed to the outside of the system during the extraction of a portion of the (poly)alkylene glycol phase for regeneration of the catalyst. The olefin phase of the upper layer in the liquid-liquid separator 43 is introduced into the distillation column 44 through the conduit 53. Under controlling the pressure of the distillation column 44, the temperature of the olefin phase, and the reflux ratio of the distillation column 44, components having a low boiling point present in the olefin phase, that is, an unreacted olefin including the branched olefin, and the alcohol are introduced into the distillation column 45 from the top of the column through the conduit 56.

According to one embodiment of the present invention, the column top pressure of the distillation column (distillation column 44 in FIG. 3) into which the olefin phase subjected to liquid-liquid separation has been introduced is suitably 0.01 to 50 kPa, 0.05 to 20 kPa, or 0.1 to 10 kPa. According to one embodiment of the present invention, the column bottom temperature of the distillation column (distillation column 44 in FIG. 3) into which the olefin phase subjected to liquid-liquid separation has been introduced is suitably 30 to 250° C., 50 to 230° C., or 70 to 200° C. According to one embodiment of the present invention, the column top temperature of the distillation column (distillation column 44 in FIG. 3) into which the olefin phase subjected to liquid-liquid separation has been introduced is suitably 30 to 230° C., 40 to 210° C., or 50 to 200° C. According to one embodiment of the present invention, the reflux ratio of the distillation column (distillation column 44 in FIG. 3) into which the olefin phase subjected to liquid-liquid separation has been introduced is suitably 0.01 to 300, 0.05 to 100, or 0.1 to 50. Under such conditions, the (poly)alkylene glycol monoalkyl ether and the (poly)alkylene glycol dialkyl ether can be efficiently separated from the other components in the liquid-liquid separator.

Further, under controlling the pressure of the distillation column 45, the temperature of the olefin phase, and the reflux ratio of the distillation column 45, at least a portion of the branched olefin, which is the component having the lowest boiling point among the components introduced into the distillation column 45, is removed through the conduit 61. This makes it possible to reduce the concentration of the branched olefin accumulated in the reaction system.

According to one embodiment of the present invention, the column top pressure of the distillation column (distillation column 45 in FIG. 3) for removing at least a portion of the branched olefin is suitably 0.01 to 50 kPa, 0.05 to 20 kPa, or 0.1 to 10 kPa. According to one embodiment of the present invention, the column bottom temperature of the distillation column (distillation column 45 in FIG. 3) for removing at least a portion of the branched olefin is suitably 30 to 230° C., 40 to 210° C., or 50 to 200° C. According to one embodiment of the present invention, the column top temperature of the distillation column (distillation column 45 in FIG. 3) for removing at least a portion of the branched olefin is suitably 30 to 200° C., 40 to 180° C., or 50 to 150° C. According to one embodiment of the present invention, the column top temperature of the distillation column (distillation column 45 in FIG. 3) for removing at least a portion of the branched olefin is preferably lower than 80° C. if the column top pressure is set to 0.9 to 1.0 kPa.

The proportion of the (poly)alkylene glycol monoalkyl ether and the (poly)alkylene glycol dialkyl ether in the liquid (liquid flowing through the conduit 56) introduced into the distillation column in the present embodiment is usually 1.0 mass % or less, or 0.5 mass % or less. According to one embodiment of the present invention, the reflux ratio of the distillation column (distillation column 45 in FIG. 3) for removing at least a portion of the branched olefin is suitably 0.01 to 300, 0.05 to 200, or 0.1 to 100.

The unreacted olefin from which at least a portion of the branched olefin has been removed and the alcohol are put into the continuous tank-type reactor 41 from the bottom of the distillation column 45 through the conduit 58 and further through the raw material supply pipe 50, and at this time, the linear olefin is replenished as necessary in an amount corresponding to the amount consumed by the reaction.

According to one embodiment of the present invention, the branched olefin ratio in the conduit 58 is controlled not to be less than 1.0 mass %, preferably 1.5 mass % or less, 1.7 mass % or less, 1.9 mass % or less, 2 mass % or less, 2.2 mass % or less, 2.4 mass % or less, 2.6 mass % or less, or 2.8 mass % or less. According to one embodiment of the present invention, the branched olefin ratio in the conduit 58 is controlled not to be more than 20 mass %, 15 mass % or more, 10 mass % or more, 9 mass % or more, 8 mass % or more, 7 mass % or more, or 6 mass % or more.

On the other hand, the (poly)alkylene glycol monoalkyl ether and the (poly)alkylene glycol dialkyl ether as a by-product are extracted from the bottom of the distillation column 44 through the conduit 57 and introduced into the distillation column 46. Under controlling the pressure of the distillation column 46, the temperature of the (poly)alkylene glycol monoalkyl ether phase, and the reflux ratio of the distillation column 46, the (poly)alkylene glycol monoalkyl ether, which is a component having a low boiling point, is extracted as a distillate from the top of the distillation column 46 through the conduit 62.

According to one embodiment of the present invention, the column top pressure of the distillation column (distillation column 46 in FIG. 3) for preparing the target (poly)alkylene glycol monoalkyl ether is suitably 10 to 3000 Pa, 20 to 1000 Pa, or 30 to 500 Pa. According to one embodiment of the present invention, the column bottom temperature of the distillation column (distillation column 46 in FIG. 3) for preparing the target (poly)alkylene glycol monoalkyl ether is suitably 50 to 350° C., 100 to 300° C., or 150 to 280° C. According to one embodiment of the present invention, the column top temperature of the distillation column (distillation column 46 in FIG. 3) for preparing the target (poly)alkylene glycol monoalkyl ether is suitably 30 to 300° C., 50 to 280° C., or 100 to 250° C. According to one embodiment of the present invention, the reflux ratio of the distillation column (distillation column 46 in FIG. 3) for preparing the target (poly)alkylene glycol monoalkyl ether is suitably 0.01 to 300, 0.05 to 200, or 0.1 to 100.

The (poly)alkylene glycol dialkyl ether extracted from the bottom of the distillation column 46 is put into the continuous tank-type reactor 41 through the conduit 59 and further through the raw material supply pipe 50. In a case where impurities such as a heavy component accumulate in the (poly)alkylene glycol dialkyl ether phase, a portion of the (poly)alkylene glycol dialkyl ether phase can be purged through the conduit 60 to remove the heavy component. When such a flow-type reaction is repeated, the (poly)alkylene glycol dialkyl ether and/or the alcohol as a by-product is converted into the (poly)alkylene glycol monoalkyl ether, whereby the (poly)alkylene glycol monoalkyl ether can be prepared from the linear olefin and the (poly)alkylene glycol with high selectivity and high efficiency.

As described above, to reduce the concentration of the branched olefin in the reaction apparatus, it is preferable to introduce a facility capable of removing at least a portion of the branched olefin. Specifically, the branched olefin has a lower boiling point than that of the linear olefin, and thus at least a portion of the branched olefin can be removed from the olefins by installing a distillation column. In the process described in FIG. 2, the distillation column for removing the branched olefin removes the branched olefin as a light boiling component by distillation from the olefin phase separated by the liquid-liquid separator. In the process described in FIG. 3, low-boiling components such as olefins and an alcohol as a by-product are separated in advance from the olefin phase separated by the liquid-liquid separator, and then the branched olefin is removed from the unreacted olefin and the alcohol as a by-product.

### EXAMPLES

The present invention will be described in more detail below by way of Examples and Comparative Examples, but is not limited to these. Note that an experimental example in which continuous operation was not performed will be described as a reference example. In Examples, the yield of a product per reaction (per passage through the reactor) was calculated in accordance with the following equation.

Yield of (poly)alkylene glycol monoalkyl ether per reaction (per passage through reactor) (abbreviated as Y-E (mol %))=(number of moles of (poly)alkylene glycol monoalkyl ether produced/number of moles of olefin supplied)×100.

### Reference Example 1

33.18 g of BEA-type zeolite available from Zeolyst (product name: CP 811E, atomic ratio of Si to Al in catalyst: 13.0, specific surface area: 656 m²/g) as a catalyst, 270 g (1.60 mol) of 1-dodecene, and 298.69 g (4.81 mol) of monoethylene glycol were put into a 1000-mL glass reactor equipped with a stirring blade and a reflux condenser, a gas phase portion was replaced with nitrogen, and then the mixture was held in nitrogen atmosphere at ordinary pressure. Note that 1-dodecene and monoethylene glycol as raw materials were sufficiently dehydrated, and the catalyst was dried at 300° C. for 3 hours before use. The inside of the reactor was separated into two phases of an olefin phase and a monoethylene glycol phase, and the catalyst was dispersed in the monoethylene glycol phase.

Then, the temperature was raised to 150° C. while the mixture was stirred at a rotational speed of 500 rpm, and the reaction was performed at the same temperature for 1 hour. Thereafter, the reaction liquid was cooled to room temperature, and products in the olefin phase and the monoethylene glycol phase were analyzed by gas chromatography. The olefin phase contained mainly unreacted dodecene and monoethylene glycol monododecyl ether, and the monoethylene glycol phase contained mainly unreacted monoethylene glycol, diethylene glycol, and water. The analytical results are shown in Table 1.

### Reference Example 2

The reaction and analysis were performed in the same manner as in Reference Example 1, except that 2-methyl-1-undecene, which is a branched olefin of C₁₂, was used instead of 1-dodecene. The results are shown in Table 1.

### Reference Example 3

The reaction and analysis were performed in the same manner as in Reference Example 1, except that dodecene, which was a mixture of linear olefins of C₁₂ (containing 16 mass % of 1-dodecene and the remaining 84 mass % is a mixture of 2-dodecene, 3-dodecene, 4-dodecene, 5-dodecene, and 6-dodecene which are inner olefins) was used instead of 1-dodecene. The results are shown in Table 1.

### Reference Example 4

The reaction and analysis were performed in the same manner as in Example 1 except that a mixture of branched olefins of C₁₂ (a mixture of 2-methyl-1-undecene, 2-methyl-2-undecene, and the like) was used instead of 1-dodecene. The results are shown in Table 1.

TABLE 1

| | Y-E (mol %) |
|---|---|
| Reference Example 1 | 25.4% |
| Reference Example 2 | 5.6% |
| Reference Example 3 | 7.5% |
| Reference Example 4 | 3.7% |

The above results revealed that the yield of monoethylene glycol monoalkyl ether was lower in a case where a branched olefin was used as a raw material (Reference Examples 2 and 4) than in a case where a linear olefin was used as a raw material (Reference Examples 1 and 3).

### Example 1

Ethylene glycol monododecyl ether was continuously produced using a continuous reaction apparatus as illustrated in FIG. 2. As the continuous tank-type reactors 11 and 12, 1000-mL continuous tank-type reactors made of stainless steel equipped with stirrers (stirring devices 11a and 12a) and band heaters for heating (heating devices 11b and 12b) were used. The continuous tank-type reactors 11 and 12 were provided with overflow lines indicated by the conduits 21 and 22. The overflow lines were arranged in such a manner that a reaction liquid flowed from the continuous tank-type reactor 11 to the continuous tank-type reactor 12 and then to the liquid-liquid separator 13 depending on a supply rate of raw materials to be supplied through the raw material supply pipe 20. As the distillation column 14, an Oldershaw distillation column having 20 stages and an inside diameter of 32 mmφ was used, and the conduit 23 was connected to the seventh stage from the top of the column. A reflux device (not illustrated) was installed at the top of the distillation column 14. In addition, a preheater (not illustrated) was installed near the connecting portion between the conduit 23 and the distillation column 14 to heat the reaction liquid supplied from the conduit 23 to the distillation column 14. As the distillation column 15, an Oldershaw distillation column having 15 stages and an inside diameter of 32 mmφ was used, and the conduit 27 was connected to the fifth stage from the top of the column. A reflux device (not illustrated) was installed at the top of the distillation column 15. In addition, a preheater (not illustrated) was installed near the connecting portion between the conduit 27 and the distillation column 15 to heat the reaction liquid supplied from the conduit 27 to the distillation column 15. As the distillation column 16, a packed column made of stainless steel and having an inside diameter of 20 mmφ and a height of 500 mm was used, and a Dickson packing made of stainless steel of 1.5 mmφ was packed as a packing. A reflux device (not illustrated) was installed at the top of the column. The conduit 28 was connected to the central portion of the distillation column 16, and a preheater (not illustrated) was installed near the connecting portion to heat the reaction liquid supplied from the conduit 28 to the distillation column 16. The distillation columns 14, 15, and 16 were each provided with a pressure reducing device, and distillation was performed under reduced pressure.

268 g (about 1.6 mol) of 1-dodecene (branched olefin content: 3 to 5 mass %), 298 g (about 4.8 mol) of monoethylene glycol, and 32.7 g of BEA-type zeolite available from PQ Corporation (product name: VALFOR CP 811BL-25, atomic ratio of Si to Al: 12.5, specific surface area: 750 m²/g) as a catalyst were put into each of the continuous tank-type reactors 11 and 12, and the stirrer was operated at a rotational speed of 600 rpm. Then, the temperature in the reactor was raised to 150° C., and thereafter the same temperature was maintained. 1-dodecene (branched olefin content: 3 to 5 mass %), monoethylene glycol, and the catalyst were supplied from the raw material supply pipe 20 to the continuous tank-type reactor 11 at supply rates of 268 g/hr, 298 g/hr, and 32.7 g/hr, respectively, to start the reaction. Note that the catalyst was previously suspended in monoethylene glycol and supplied. The reaction liquid discharged from the reactor 11 was transferred to the reactor 12 through the conduit 21 to continue the reaction, and the reaction liquid discharged from the reactor 12 was transferred to the liquid-liquid separator 13 through the conduit 22 to be separated into a monoethylene glycol phase containing the catalyst and an olefin phase containing monoethylene glycol monododecyl ether. The monoethylene glycol phase was recycled to the continuous tank-type reactor 11 through the conduit 24. At this time, 5 mass % of the flow rate was purged from the conduit 25 to the outside of the system.

On the other hand, the olefin phase was supplied to the distillation column 14 through the conduit 23. The operating conditions of the distillation column 14 were as follows: the column top pressure was 1.3 kPa, the column bottom temperature was 100° C., the column top temperature was 80° C., and the reflux ratio was 50. The distillate from the distillation column 14 was mainly branched C₁₂ olefins and a small amount of linear dodecene and was removed through the conduit 31. The bottom liquid of the distillation column 14 was supplied to the distillation column 15 through the conduit 27. The branched olefin ratio (the mass of C₁₂ branched olefin relative to the mass of dodecenes (C₁₂ linear olefin and C₁₂ branched olefin)) of the bottom liquid supplied from the distillation column 14 to the distillation column 15 was controlled to be 2 to 5 mass %.

The operating conditions of the distillation column 15 were as follows: the column top pressure was 1.3 kPa, the column bottom temperature was 170° C., the column top temperature was 88° C., and the reflux ratio was 0.5. The distillate of the distillation column 15 was mainly unreacted isomerized linear dodecene and was recycled to the reactor 11 through the conduit 26. It was confirmed that the mass of the C₁₂ branched olefin (branched olefin ratio) relative to the mass sum of dodecenes (C₁₂ linear olefin and C₁₂ branched olefin) in the distillate flowing through the conduit 26 was controlled to be within a range from 3 to 5 mass % at 1000 to 3000 hours after the start of operation.

The bottom liquid of the distillation column 15 was supplied to the distillation column 16 through the conduit 28. The operating conditions of the distillation column 16 were as follows: the column top pressure was 270 Pa, the column bottom temperature was 220° C., the column top temperature was 150° C., and the reflux ratio was 0.5. The distillate from the distillation column 16 was mainly monoethylene glycol monododecyl ether, which was the target product, and was recovered as a product through the conduit 32. The bottom liquid of the distillation column 16 was mainly monoethylene glycol didodecyl ether, and was recycled to the continuous tank-type reactor 11 through the conduit 29. Note that in the present Example, a portion of the bottom liquid of the distillation column 16 was not purged through the conduit 30.

After the reaction was started, to fit flow rates of the recovered raw material to be recycled through the conduits 24, 26, and 29 and the catalyst, supply amounts of new (fresh) raw materials (1-dodecene, monoethylene glycol) to be supplied from the raw material supply pipe 20 and a new or regenerated catalyst were adjusted to perform control in such a manner that in the composition of raw materials to be supplied to the continuous tank-type reactor 11, a molar ratio of monoethylene glycol/dodecenes was 3/1, the amount of the catalyst was 10 mass % in the monoethylene glycol phase, and the flow rate of supplied liquid was the liquid hourly space velocity (LHSV) in the continuous tank-type reactor 11 of 1 hr⁻¹.

From 1000 hours to 3000 hours after the start of operation of the continuous reaction apparatus under the above operating conditions, the branched olefin ratio of the dodecenes to be supplied to the reactor 11 was controlled to be 3 to 5 mass % during the operation time. The yield (Y-E (mol %)) of the monoethylene glycol monododecyl ether recovered from the conduit 32 through the reactor 12 relative to the dodecenes to be supplied to the reactor 11 was 10.2% at 1000 hours and 10.0% at 3000 hours, and transitioned within a range of 10%±1.2% from 1000 hours to 3000 hours. During this time, the overall process yield (olefin utilization efficiency) of the target monoethylene glycol monododecyl ether relative to 1-dodecene fed (supplied) from the raw material supply pipe 20 was 88±2 mol %. The yield of monoethylene glycol monododecyl ether per unit time was 323 g/hr.

### Comparative Example 1

An experiment was performed in the same manner as in Example 1, except that after the distillation column 14 was operated under the conditions of Example 1 for up to 1000 hours, the operation of the distillation column 14 was stopped (the distillation column 14 was used as a simple bypass to the distillation column 15).

The mass of the C₁₂ branched olefin (branched olefin ratio) relative to the mass sum of the dodecenes (the C₁₂ linear olefin and the C₁₂ branched olefin) in the distillate flowing through the conduit 26 was increased from 4.5 mass % to 30 mass % with time from 1000 hours to 2000 hours after the start of operation, and was not able to be controlled not to exceed 20 mass %.

In addition, the branched olefin ratio of the dodecenes to be supplied to the reactor 11 was increased from 4.5 mass % to 30 mass % over the operation time from 1000 hours to 2000 hours after the start of operation of the continuous reaction apparatus under the above operation conditions. The yield (Y-E (mol %)) of monoethylene glycol monododecyl ether recovered from the conduit 32 through the reactor 12 relative to the dodecenes to be supplied to the reactor 11 was 10.0% at 1000 hours and 6.4% at 2000 hours, and was clearly decreased from 1000 hours to 2000 hours. The operation was performed to maintain the retention time of the reaction liquid in the reactor and the liquid level of the reaction liquid in the reactor, and thus the supply amount of the raw materials to be introduced from the raw material supply pipe 20 was reduced as compared with the initial introduction amount, and the feed amount per hour at the time of 2000 hours was reduced to about ⅔ (180 g/hr as 1-dodecene) of the initial amount. The overall process yield (olefin utilization efficiency) of the target monoethylene glycol monododecyl ether relative to 1-dodecene fed from the raw material supply pipe 20 was 89 mol % at the time of 2000 hours. The yield of monoethylene glycol monododecyl ether per unit time at the time of a lapse of 2000 hours was 220 g/hr.

### Example 2

Ethylene glycol monododecyl ether was continuously produced using a continuous reaction apparatus as illustrated in FIG. 3. As the continuous tank-type reactors 41 and 42, 1000-mL continuous tank-type reactors made of stainless steel equipped with stirrers (stirring devices 41a and 42a) and band heaters for heating (heating devices 41b and 42b) were used. The continuous tank-type reactors 41 and 42 were provided with overflow lines indicated by the conduits 51 and 52. The overflow lines were arranged in such a manner that a reaction liquid flowed from the continuous tank-type reactor 41 to the continuous tank-type reactor 42 and then to the liquid-liquid separator 43 depending on a supply rate of raw materials to be supplied through the raw material supply pipe 50. As the distillation column 44, an Oldershaw distillation column having 20 stages and an inside diameter of 32 mmφ was used, and the conduit 53 was connected to the seventh stage from the top of the column. A reflux device (not illustrated) was installed at the top of the distillation column 44. In addition, a preheater (not illustrated) was installed near the connecting portion between the conduit 53 and the distillation column 44 to heat the reaction liquid supplied from the conduit 53 to the distillation column 44. As the distillation column 45, an Oldershaw distillation column having 15 stages and an inside diameter of 32 mmφ was used, and the conduit 56 was connected to the fifth stage from the top of the column. A reflux device (not illustrated) was installed at the top of the distillation column 45. In addition, a preheater (not illustrated) was installed near the connecting portion between the conduit 56 and the distillation column 45 to heat the reaction liquid supplied from the conduit 56 to the distillation column 45. As the distillation column 46, a packed column made of stainless steel and having an inside diameter of 20 mmφ and a height of 500 mm was used, and a Dickson packing made of stainless steel of 1.5 mmφ was packed as a packing. A reflux device (not illustrated) was installed at the top of the column. The conduit 57 was connected to the central portion of the distillation column 46, and a preheater (not illustrate) was installed near the connecting portion to heat the reaction liquid supplied from the conduit 57 to the distillation column 46. The distillation columns 44, 45, and 46 were each provided with a pressure reducing device, and distillation was performed under reduced pressure.

268 g of 1-dodecene (branched olefin content: 3 mass % to 5 mass %), 298 g of monoethylene glycol, and 32.7 g of BEA-type zeolite available from PQ Corporation (product name: VALFOR CP 811BL-25, atomic ratio of Si to Al: 12.5, specific surface area: 750 m²/g) as a catalyst were put into each of the continuous tank-type reactors 41 and 42, and the stirrer was operated at a rotational speed of 600 rpm. Then, the temperature in the reactor was raised to 150° C., and thereafter the same temperature was maintained. 1-dodecene (branched olefin content: 3 mass % to 5 mass %), monoethylene glycol, and the catalyst were supplied from the raw material supply pipe 50 to the continuous tank-type reactor 41 at supply rates of 268 g/hr, 298 g/hr, and 32.7 g/hr, respectively, to start the reaction. Note that the catalyst was previously suspended in monoethylene glycol and supplied. The reaction liquid was transferred to the liquid-liquid separator 43 through the conduit 52 and separated into a monoethylene glycol phase containing the catalyst and an olefin phase containing monoethylene glycol monododecyl ether. The monoethylene glycol phase was recycled to the continuous tank-type reactor 41 through the conduit 54. At this time, 5 mass % of the flow rate was purged from the conduit 55 to the outside of the system.

On the other hand, the olefin phase was supplied to the distillation column 44 through the conduit 53. The operating conditions of the distillation column 44 were as follows: the column top pressure was 1.5 kPa, the column bottom temperature was 100° C., the column top temperature was 90° C., and the reflux ratio was 0.5. The distillate from the distillation column 44 was mainly branched C₁₂ olefins and linear dodecenes and was supplied to the distillation column 45 through the conduit 56. The bottom liquid of the distillation column 44 was supplied to the distillation column 46 through the conduit 57. The operating conditions of the distillation column 45 were as follows: the column top pressure was 1.0 kPa, the column bottom temperature was 80° C., the column top temperature was 70° C., and the reflux ratio was 50. The distillate from the distillation column 45 was mainly branched C₁₂ olefins and a small amount of linear dodecene and was removed through the conduit 61. The bottom liquid of the distillation column 45 was recycled to the reactor 41 through the conduit 58. It was confirmed that the mass of the C₁₂ branched olefin (branched olefin ratio) relative to the mass sum of dodecenes (C₁₂ linear olefin and C₁₂ branched olefin) in the distillate flowing through the conduit 58 from the distillation column 45 to be recycled (supplied) to the reactor 41 was controlled to be within a range from 2 to 5 mass % from 1000 to 2000 hours after the start of operation.

The operating conditions of the distillation column 46 were as follows: the column top pressure was 400 Pa, the column bottom temperature was 240° C., the column top temperature was 140° C., and the reflux ratio was 0.5. The distillate from the distillation column 46 was mainly monoethylene glycol monododecyl ether, which was the target product, and was recovered as a product through the conduit 62. The bottom liquid of the distillation column 46 was mainly monoethylene glycol didodecyl ether and was recycled to the continuous tank-type reactor 41 through the conduit 59. Note that in the present Example, a portion of the bottom liquid of the distillation column 46 was not purged through the conduit 60.

After the reaction was started, to fit flow rates of the recovered raw material to be recycled through the conduits 54, 58, and 59 and the catalyst, supply amounts of new raw materials (1-dodecene, monoethylene glycol) to be supplied from the raw material supply pipe 50 and a new or regenerated catalyst were adjusted to perform control in such a manner that in the composition of raw materials to be supplied to the continuous tank-type reactor 41, a molar ratio of monoethylene glycol/dodecenes was 3/1, the amount of the catalyst was 10 mass % in the monoethylene glycol phase, and the flow rate of supplied liquid was the liquid hourly space velocity (LHSV) in the continuous tank-type reactor 41 of 1 hr⁻¹.

In addition, the branched olefin ratio in the dodecenes to be supplied to the reactor 41 was controlled to be 2 to 5 mass % over the operation time from 1000 to 2000 hours after the start of operation of the continuous reaction apparatus under the above operation conditions. The yield (Y-E (mol %)) of the monoethylene glycol monododecyl ether recovered from the conduit 62 through the reactor 42 relative to the dodecenes to be supplied to the reactor 41 was 9.9% at 1000 hours and 10.0% at 2000 hours, and transitioned within a range of 10%+1.1% from 1000 hours to 3000 hours. At this time, the overall process yield (olefin utilization efficiency) of the target monoethylene glycol monododecyl ether relative to 1-dodecene fed from the raw material supply pipe 50 was 87±2 mol %. The yield of monoethylene glycol monododecyl ether per unit time was 320 g/hr.

### Example 3

An experiment was performed in the same manner as in Example 2, except that after the distillation column 45 was operated under the conditions of Example 2 for up to 1000 hours, the column top temperature of the distillation column 45 was set to 80° C. to further decrease the branched olefin ratio, thereby decreasing the branched olefin ratio in the dodecenes to be supplied to the reactor 41 as compared with Example 2. The mass of the C₁₂ branched olefin (branched olefin ratio) relative to the mass sum of the dodecenes (C₁₂ linear olefin and C₁₂ branched olefin) in the distillate flowing through the conduit 58 was decreased from 4.0 mass % to 1.5 mass % over time from 1000 hours to 1500 hours after the start of operation.

In addition, the branched olefin ratio of the dodecenes to be supplied to the reactor 41 was decreased from 4.0 mass % to 1.7 mass % over the operation time from 1000 hours to 1500 hours after the start of operation of the continuous reaction apparatus under the above operation conditions. The yield (Y-E (mol %)) of monoethylene glycol monododecyl ether recovered from the conduit 62 through the reactor 42 relative to the dodecenes to be supplied to the reactor 41 was 9.9% at 1000 hours and 10.6% at 1500 hours, and was slightly improved from 1000 hours to 1500 hours.

However, the olefin as the raw material was removed together with the removal of the branched olefin, and thus the utilization efficiency of the olefin was lowered, and the overall process yield (utilization efficiency of olefin) of the target monoethylene glycol monododecyl ether relative to the total 1-dodecene fed from the raw material supply pipe 50 was 81 mol % at the time of 1500 hours. The yield of monoethylene glycol monododecyl ether per unit time was 290 g/hr.

As described above, it is found that the branched olefin ratio in the recovered raw materials is preferably controlled not to be 1.5 mass % or less.

From the above Examples and Comparative Examples, when the branched olefin concentration was increased, the yield of monoethylene glycol monododecyl ether before and after the reactor was decreased, and the overall process yield (utilization efficiency of olefin) was almost unchanged, but the yield per hour was significantly decreased. On the other hand, when the branched olefin concentration was decreased, the yield of monoethylene glycol monododecyl ether before and after the reactor was increased, but the loss of the raw materials in distillation was large and the overall process yield (utilization efficiency of olefin) was decreased, and the yield per hour was also decreased.

### Reference Example 5

The reaction and analysis were performed in the same manner as in Reference Example 3 except that the catalyst deteriorated by the reaction for 3000 hours in Example 1 was recovered and used as the catalyst. The results are shown in Table 2.

### Reference Example 6

The reaction and analysis were performed in the same manner as in Reference Example 4 except that the catalyst deteriorated by the reaction for 3000 hours in Example 1 was recovered and used as the catalyst. The results are shown in Table 2.

TABLE 2

| | Y-E (mol %) |
|---|---|
| Reference Example 5 | 8.0% |
| Reference Example 6 | 0.7% |

From the above results, the yield of monoethylene glycol monoalkyl ether per reaction was clearly decreased in a case where the reaction was performed using the deteriorated catalyst and a branched olefin as a raw material (Reference Example 6) as compared with a case where the reaction was performed using the deteriorated catalyst and a linear olefin as a raw material (Reference Example 5). In particular, when Reference Example 4 and Reference Example 6 were compared with each other, the decrease in yield per reaction due to the deterioration of the catalyst was very sharp in a case where the branched olefin was used as the raw material, and it was found that the decrease in yield per reaction of monoethylene glycol monododecyl ether (in passing through the reactor once) in Comparative Example 1 was mostly caused by the branched olefin.

### INDUSTRIAL APPLICABILITY

The (poly)alkylene glycol monoalkyl ether produced by the present invention is useful as a raw material for a surfactant, and the present invention can provide a method for producing a (poly)alkylene glycol monoalkyl ether useful for reduction of resources and energy consumption based on the viewpoint of reduction of load on the global environment, conservation of resources, carbon neutrality, SDGs (sustainable development goals), and the like.

### REFERENCE SIGNS LIST

1: Batch reactor
1a: Stirring device
1b: Heating device
2: Distillation column
3: Conduit
4: Raw material supply pipe
5, 6: Extraction pipe
11, 12, 41, 42: Continuous tank-type reactor
11a, 12a, 41a, 42a: Stirring device
11b, 12b, 41b, 42b: Heating device
13, 43: Liquid-liquid separator
14, 15, 16, 44, 45, 46: Distillation column
20, 50: Raw material supply pipe
21 to 32, 51 to 62: Conduit.

This application is based on Japanese Patent Application No. 2022-169504 filed on Oct. 21, 2022, the disclosure of which is incorporated herein by reference in its entirety.

1. A method for producing a (poly)alkylene glycol monoalkyl ether comprising reacting an olefin and a (poly)alkylene glycol in a reactor in a presence of a catalyst, the method comprising recovering at least a portion of raw materials used for the production and using the recovered raw materials again as a raw material,
wherein
at least one of the recovered raw materials contains an olefin, and
a mass of a branched olefin relative to a mass sum of the branched olefin and a linear olefin contained in the recovered raw materials is controlled not to exceed 20 mass %.

2. The method according to claim 1, wherein the olefin is an olefin having 6 or more and 20 or less carbon atoms.

3. The method according to claim 2, wherein the recovered raw materials contain an olefin from which a branched olefin has been separated by distillation.

4. The method according to claim 1, wherein at least one of the recovered raw materials contains a (poly)alkylene glycol.

5. The method according to claim 1, wherein a solid acid catalyst is used as the catalyst.

6. The method according to claim 5, wherein a crystalline metallosilicate is used as the solid acid catalyst.

7. The method according to claim 1, further comprising controlling the mass of the branched olefin relative to the mass sum of the branched olefin and the linear olefin contained in the recovered raw materials not to be 1.5 mass % or less.
